(19) United States
(12) Patent Application Publication
(10) Pub. No.: US 2025/0032251 A1
(43) Pub. Date: Jan. 30, 2025

(54) PROSTHETIC HEART VALVE DELIVERY ASSEMBLY

(71) Applicant: Medtronic, Inc., Minneapolis, MN (US)

(72) Inventor: Marc A. ANDERSON, Barna (IE)

(73) Assignee: Medtronic, Inc., Minneapolis, MN (US)

(21) Appl. No.: 18/616,570

(22) Filed: Mar. 26, 2024

Related U.S. Application Data
(60) Provisional application No. 63/529,119, filed on Jul. 26, 2023.

(51) Int. Cl. A61F 2/24

(52) U.S. Cl. CPC A61F 2/2433; A61F 2/2412

(57) ABSTRACT

A transcatheter heart valve delivery assembly includes a first shaft portion including a first wall surrounding a first chamber. A second shaft portion is attached to a distal end of the first shaft portion and includes a second wall surrounding a second chamber. A funnel portion is attached to a distal end of the second shaft portion and includes a funnel wall surrounding a funnel chamber. The funnel portion moves between a radially-compressed position, in which the funnel chamber includes a first diameter that is less than or equal to a diameter of the second chamber, and a radially-expanded position, in which the funnel chamber includes a second diameter that is greater than the first diameter. The funnel portion is biased into the radially-compressed position. Methods of recapturing a heart valve prosthesis are provided.

### CROSS-REFERENCE TO RELATED APPLICATION

This application claims the benefit of U.S. Provisional Patent Application Ser. No. 63/529,119, filed Jul. 26, 2023, the entire content of which is incorporated herein by reference.

### FIELD

The present disclosure relates generally to a prosthetic heart valve assembly and, more particularly, to a delivery assembly for delivering a heart valve prosthesis.

### BACKGROUND

It is known to provide a prosthetic heart valve assembly for implanting a heart valve prosthesis within a target site of the vasculature of a patient. The heart valve prosthesis can be moved from a radially-collapsed position to a radially-expanded position. However, recapture of the heart valve prosthesis can be difficult.

### SUMMARY

The following presents a simplified summary of the disclosure to provide a basic understanding of some aspects described in the detailed description.

In aspects, a transcatheter heart valve delivery assembly is provided for delivering a heart valve prosthesis to a treatment site. The transcatheter heart valve delivery assembly comprises a first shaft portion comprising a first wall surrounding a first chamber and extending along an axis. The first wall comprises a first durometer value. The transcatheter heart valve delivery assembly comprises a second shaft portion attached to a distal end of the first shaft portion and comprising a second wall surrounding a second chamber. The second shaft portion extends along the axis. The second wall comprises a second durometer value that is less than the first durometer value. The transcatheter heart valve delivery assembly comprises a funnel portion attached to a distal end of the second shaft portion and comprising a funnel wall surrounding a funnel chamber. The funnel portion extends along the axis and can move between a radially-compressed position, in which the funnel chamber comprises a first diameter that is less than or equal to a diameter of the second chamber, and a radially-expanded position, in which the funnel chamber comprises a second diameter that is greater than the first diameter. The funnel wall comprises a third durometer value that is less than the second durometer value. The funnel portion is biased into the radially-compressed position and configured to receive and compress the heart valve prosthesis within the funnel chamber.

In aspects, the funnel portion comprises one or more of a polyether-based thermoplastic polyurethane material or a thermoplastic elastomer material.

In aspects, the funnel wall comprises a funnel wall thickness within a range from about 0.3 millimeters to about 0.6 millimeters.

In aspects, the first diameter is within a range from about 5 millimeters to about 10 millimeters and the second diameter is within a range from about 12 millimeters to about 20 millimeters.

In aspects, the funnel portion comprises a length between a distal funnel end of the funnel portion and a proximal funnel end of the funnel portion that is within a range from about 5 millimeters to about 10 millimeters.

In aspects, the second shaft portion is reinforced with one or more of a frame or a coil.

In aspects, the second wall comprises a second wall thickness within a range from about 0.4 millimeters to about 0.8 millimeters.

In aspects, the second chamber comprises a second chamber diameter within a range from about 5 millimeters to about 10 millimeters.

In aspects, the first shaft portion is reinforced with one or more of a frame, a braid, or a coil.

In aspects, the first chamber, the second chamber, and the funnel chamber extend coaxially and contiguously through the first shaft portion, the second shaft portion, and the funnel portion, the first chamber, the second chamber, and the funnel chamber configured to receive at least a portion of the delivery assembly through the first shaft portion, the second shaft portion, and the funnel portion.

In aspects, a transcatheter heart valve delivery assembly is provided for delivering a heart valve prosthesis to a treatment site. The transcatheter heart valve delivery assembly comprises a first shaft portion comprising a first wall surrounding a first chamber. The first wall comprises a first durometer value. The first shaft portion is configured to receive a first portion of the delivery assembly within the first chamber. The transcatheter heart valve delivery assembly comprises a second shaft portion attached to a distal end of the first shaft portion and comprising a second wall surrounding a second chamber. The second wall comprises a second durometer value that is less than the first durometer value. The second shaft portion is configured to receive a second portion of the delivery assembly within the second chamber. The transcatheter heart valve delivery assembly comprises a funnel portion attached to a distal end of the second shaft portion and comprising a funnel wall surrounding a funnel chamber. The funnel portion is configured to move between a radially-compressed position, in which a distal end of the funnel portion comprises a first diameter, and a radially-expanded position, in which the distal end of the funnel portion comprises a second diameter that is greater than the first diameter. The funnel wall comprises a third durometer value that is less than the second durometer value. The funnel portion is biased into the radially-compressed position.

In aspects, the funnel portion comprises one or more of a polyether-based thermoplastic polyurethane material or a thermoplastic elastomer material.

In aspects, the funnel wall comprises a funnel wall thickness within a range from about 0.3 millimeters to about 0.6 millimeters.

In aspects, the second shaft portion comprises one or more of a thermoplastic elastomer material or a thermoplastic polyurethane elastomer.

In aspects, the second wall comprises a second wall thickness within a range from about 0.4 millimeters to about 0.8 millimeters.

In aspects, methods of recapturing a heart valve prosthesis comprise deploying the heart valve prosthesis at a treatment site. Methods comprise positioning a funnel portion of a heart valve implant recapture apparatus adjacent to a valve end of the heart valve prosthesis with the funnel portion in a radially-compressed position. Methods comprise moving one or more of the funnel portion relative to the heart valve prosthesis or the heart valve prosthesis relative to the funnel portion such that the funnel portion receives the heart valve prosthesis within the funnel chamber and the funnel portion moves from the radially-compressed position to a radially-expanded position. Methods comprise applying a radial force from the funnel portion to the heart valve prosthesis to radially compress the heart valve prosthesis.

In aspects, moving the funnel portion comprises applying an axial force to the funnel portion by a second shaft portion that is attached to the funnel portion. The funnel portion comprises a durometer value that is less than the second shaft portion.

In aspects, methods can comprise applying an outward radial force from the heart valve prosthesis to the funnel portion to move the funnel portion from the radially-compressed position to a radially-expanded position.

In aspects, a diameter of the funnel chamber changes from the radially-compressed position to the radially-expanded position.

In aspects, the funnel portion is attached to a shaft portion that comprises a different material than the funnel portion.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates example aspects of a transcatheter heart valve prosthesis in accordance with aspects of the disclosure;

FIG. 2 illustrates a top-down view of the transcatheter heart valve prosthesis in accordance with aspects of the disclosure;

FIG. 3 illustrates a side view of a delivery assembly for delivering the transcatheter heart valve prosthesis in accordance with aspects of the disclosure;

FIG. 4 illustrates a side view of the delivery assembly for delivering the transcatheter heart valve prosthesis in accordance with aspects of the disclosure;

FIG. 5 illustrates an introducer sheath in accordance with aspects of the disclosure;

FIG. 6 schematically illustrates a side view of a transcatheter heart valve prosthesis and a recapture apparatus;

FIG. 7 illustrates the recapture apparatus in accordance with aspects of the disclosure;

FIG. 8 illustrates the recapture apparatus in accordance with aspects of the disclosure;

FIG. 9 illustrates the recapture apparatus in accordance with aspects of the disclosure; and

FIG. 10 illustrates the recapture apparatus in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed, collapsed, or constricted delivery configuration to an expanded deployed configuration or vice versa. Non-exhaustive exemplary self-expanding materials include stainless steel, a pseudo-clastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially collapsed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

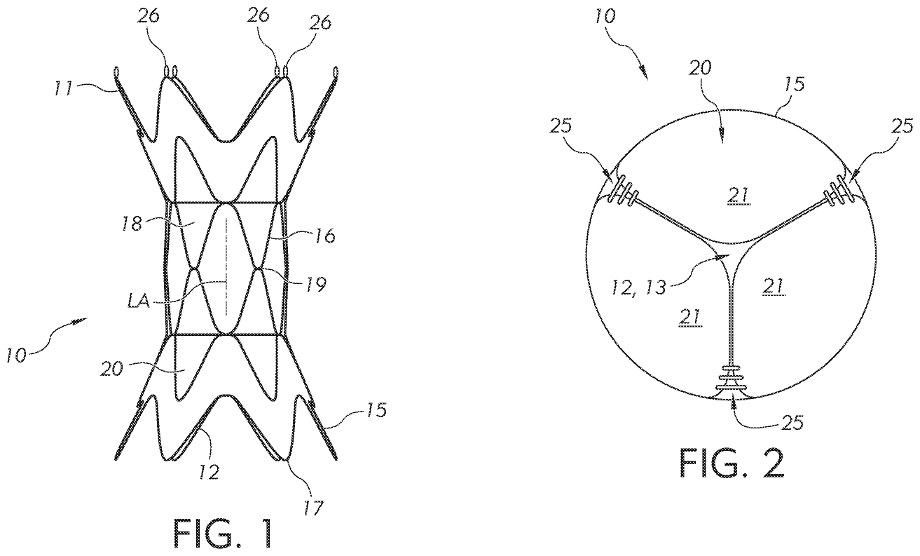

FIGS. 1 and 2 illustrate an example transcatheter heart valve prosthesis 10. The delivery assemblies described herein may be used with the transcatheter heart valve prosthesis 10 and/or other transcatheter heart valve prostheses. The transcatheter heart valve prosthesis 10 is illustrated to facilitate description of the disclosure. The following description of the transcatheter heart valve prosthesis 10 is merely exemplary in nature and is not intended to limit the invention or the application and uses of the invention.

FIGS. 1 and 2 illustrate a side view and a top/end view, respectively, of the transcatheter heart valve prosthesis 10. The transcatheter heart valve prosthesis 10 includes a radially-expandable frame or stent 15 and a prosthetic valve 20. The frame 15 of the transcatheter heart valve prosthesis 10 supports the prosthetic valve 20 within an interior of the frame 15. In the example transcatheter heart valve prosthesis 10 shown in FIGS. 1 and 2, the frame 15 is self-expandable. However, this is not meant to be limiting, and the frame 15 can be balloon-expandable or mechanically expandable in other embodiments. In some embodiments, the transcatheter heart valve prosthesis 10 may be delivered to and implanted at a treatment site within a patient to replace any of an aortic valve, a pulmonic valve, a mitral valve, and a tricuspid valve. The valve to be replaced may be a native valve or a previously-implanted prosthetic valve, such as a failed surgical replacement valve or a failed transcatheter valve.

The prosthetic valve 20 includes at least one leaflet 21 disposed within and secured to the frame 15. In the embodiment shown in FIGS. 1 and 2, the prosthetic valve 20 includes exactly three leaflets 21, as shown in FIG. 2. However, this is not meant to be limiting, as the prosthetic valve 20 may include more or fewer leaflets 21. The valve leaflets 21 open and close to regulate flow through the transcatheter heart valve prosthesis 10.

As shown in FIG. 1, the transcatheter heart valve prosthesis 10 includes an inflow end 11 and an outflow end 12. The prosthetic leaflets 21 are attached to the frame 15 at commissures 25 such that when pressure at the inflow end 11 exceeds pressure at the outflow end 12, the prosthetic leaflets 21 open to allow blood flow through the heart valve prosthesis 10 from the inflow end 11 to the outflow end 12. When the pressure at the outflow end 12 exceeds pressure at the inflow end 11, the prosthetic leaflets 21 close to prevent blood flow from the outflow end 12 to the inflow end 11. Accordingly, the at least one leaflet (e.g., the prosthetic leaflets 21) can be attached to the plurality of struts 16, for example, by being directly attached to the plurality of struts 16 at the commissures 25, or by being indirectly attached to the plurality of struts 16, for example, by being attached to a skirt, a commissure bracket, or other structure (e.g., mechanical actuator) that is attached to the plurality of struts 16.

The frame 15 of the transcatheter heart valve prosthesis 10 further includes a plurality of struts 16 that are arranged to form a plurality of openings or cells 18 arranged circumferentially around a longitudinal axis LA of the transcatheter heart valve prosthesis 10 and longitudinally to form a tubular structure defining a central lumen of the transcatheter heart valve prosthesis 10. For example, the frame 15 can extend along the longitudinal axis LA between the inflow end 11 and the outflow end 12. The frame 15 is configured to secure the prosthetic valve 20 within the central lumen of the frame 15 and to secure the transcatheter heart valve prosthesis 10 in place in the vasculature of the patient. The struts 16 are defined herein as the elongated wire segments of the frame 15. Struts 16 come together to form crowns 17 or nodes 19, as can be seen in FIG. 1. In aspects, attachment members 26 (e.g., loops) can be located at the inflow end 11 of the heart valve prosthesis 10, for example, at the crowns. The frame 15 of the heart valve prosthesis 10 includes a plurality of cells 18 defined as the spaces between the plurality of crowns 17, the plurality of nodes 19, and the plurality of struts 16. The frame 15, and, thus, the plurality of struts 16, can be adjustable between a radially-collapsed position and a radially-expanded position.

In the example embodiment shown in FIG. 1, the plurality of cells 18 may be diamond-shaped. In the example embodiment shown, the plurality of cells include a plurality of first cells 18 and, in aspects, access cells. In particular, the access cells may be larger than the first cells 18 and can provide access to one or more coronary arteries when the transcatheter heart valve prosthesis 10 is implanted in the patient. The access cells can have an enlarged area relative or compared to the first cells 18. Although not shown, in some embodiments the transcatheter heart valve prosthesis 10 may include an outer skirt extending circumferentially around an outer circumference of the stent 15 at or near the inflow end 11 to prevent paravalvular leakage of blood around the outside of the transcatheter heart valve prosthesis 10 once implanted in the patient.

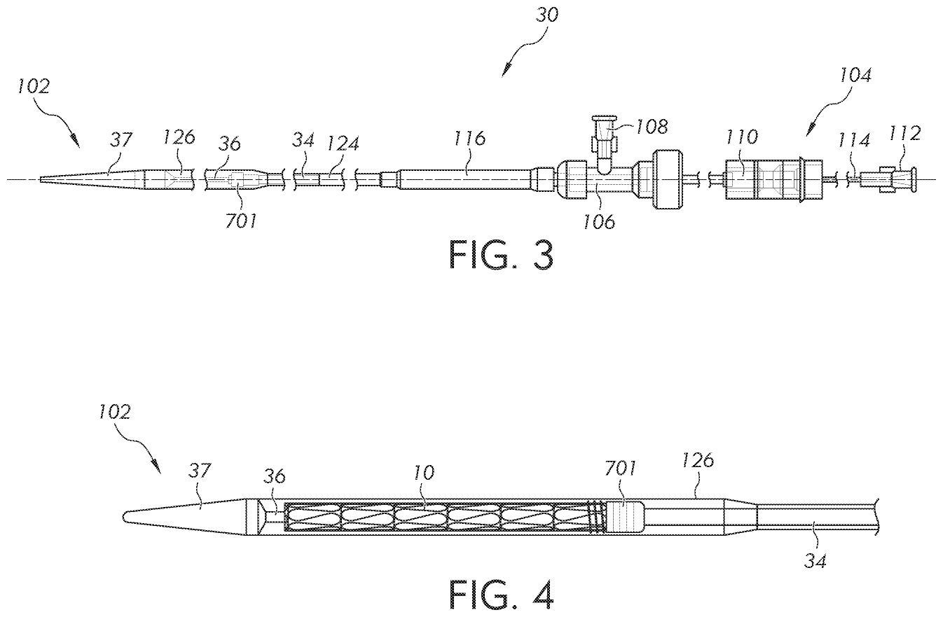

FIG. 3 illustrates an example delivery assembly 30 for a transcatheter heart valve implant apparatus having and extending between a distal end 102 and a proximal end 104. The distal end 102 can be used to load and deliver the heart valve prosthesis 10 (e.g., illustrated in FIG. 4). The proximal end 104 can comprise components, for example, those found in other catheter delivery systems for controlling transcatheter delivery of the heart valve prosthesis 10 through the vasculature of the patient and for controlling deployment of the heart valve prosthesis 10 at the target site. In aspects, the components at the proximal end 104 may comprise, for example, a first rotating homeostasis valve 106, a side access port 108, a second rotating homeostasis valve 110, and a guide member 112. The first rotating homeostasis valve 106 can comprise latex and can form a fluid seal to limit blood or other fluid from leaking out of the delivery assembly 30 at the proximal end 104 or entry site into a patient. The side access port 108 is provided to inject contrast media or saline, for example, into the delivery assembly 30. The second rotating homeostasis valve 110 may be similar to the first rotating homeostasis valve 106 and can limit blood or other fluid from leaking back through the delivery assembly 30. In addition, the second rotating homeostasis valve 110 can allow wires, devices, and fluid to pass through to aid in the preparation, delivery, and deployment of the heart valve prosthesis 10. Furthermore, the second rotating homeostasis valve 110 can control the components of the distal end 102. For example, by manipulating (e.g., rotating) a portion of the second rotating homeostasis valve 110, a physician can control components of the distal end 102. The first and second homeostasis valves 106, 110 can be compatible with catheter-laboratory components and, unless otherwise noted, the delivery assembly 30 is not intended to be limited to the specific components and features disclosed.

In aspects, the transcatheter delivery assembly 30 can be inserted into a vessel or artery of a patient, for example, the femoral artery. The proximal end 104 can extend outside of the patient, for example, in the groin area, while the distal end 102 may be delivered intravascularly to an area at or near a pulmonary valve inside the body. However, insertion of the transcatheter delivery assembly 30 in other areas of the body are also contemplated. The transcatheter delivery assembly 30 can include one or more axial lumens to pass items (e.g., guidewires, valve prosthesis, contrast media, other catheters, etc.) through the transcatheter delivery assembly 30.

In aspects, the delivery assembly 30 can comprise an inner shaft member 36 comprising at least one guidewire lumen. As used herein, a lumen comprises a cavity or hollow bore that extends through a structure, for example, the inner shaft member 36. The guide member 112 can be attached to a proximal shaft portion 114 of the the inner shaft member 36 at the proximal end 104. In aspects, the inner shaft member 36 can extend partially or completely along an entire length of the transcatheter delivery assembly 30 and may be slid or moved through other components of the transcatheter delivery assembly 30. As explained herein, a plurality of guidewires can extend through the inner shaft member 36 and can be used to guide the heart valve prosthesis 10 to a desired location. The transcatheter delivery assembly 30 can comprise an outer shaft 116 that is located between the distal end 102 and the proximal end 104, with the inner shaft member 36 extending through the outer shaft 116. The outer shaft 116 can limit blood from leaking around the transcatheter delivery assembly 30 and provides a relatively smooth, flexible length to enable the delivery assembly 30 to traverse through the vasculature of the patient.

In aspects, a tapered tip 37 can be attached to the inner shaft member 36 at the distal end 102. The tip 37 comprises a tapered shape to case the passage of the delivery assembly 30 into and through the vasculature. In aspects, a holding catheter 34 can be attached to the heart valve prosthesis 10. The holding catheter 34 can comprise a centrally located lumen surrounding the inner shaft member 36. The holding catheter 34 can slide relative to the inner shaft member 36 and may be controlled by the second rotating homeostasis valve 110 at the proximal end 104 of the delivery assembly 30. In aspects, a portion of the second rotating homeostasis valve 110 can be rotated or otherwise manipulated in order to rotate the holding catheter 34 or move the holding catheter 34 proximally and distally as desired. In aspects, a coil 701 can be attached to the distal end of the holding catheter 34. In this way, movement of the holding catheter 34, as controlled by the second rotating homeostasis valve 110, can control movement or rotation of the coil 701. In aspects, the holding catheter 34 can be surrounded by a reinforcement layer 124, which can be attached or otherwise bonded to the holding catheter 34 and can serve to reinforce the holding catheter 34. The coil 701 can be removably attached to the valve prosthesis 10, for example, by engaging the attachment members 26 (e.g., illustrated in FIG. 1). As such, the coil 701 can allow for loading of the valve prosthesis 10, holding of the valve prosthesis 10 during delivery, and release of the valve prosthesis 10 upon reaching a treatment site. In aspects, rotation of the coil 701 can cause the valve prosthesis 10 to detach from the coil 701. The delivery assembly 30 can further comprise an outer sheath 126 that may comprise a low friction and flexible material, such as polytetrafluoroethylene (PTFE), polyurethane, silicone, or polyethylene. The outer sheath 126 can be sized and shaped to receive components near the distal end 102, for example, the inner shaft member 36, the coil 701, the valve prosthesis 10, etc.

FIG. 4 illustrates the distal end 102 of the delivery assembly 30. In aspects, the valve prosthesis 10 can be in a radially-compressed position during intraluminal delivery to a treatment site. In the radially-compressed position, the valve prosthesis 10 can be removably attached to the coil 701 and circumferentially surrounded by the outer sheath 126. The valve prosthesis 10 can extend coaxially with the inner shaft member 36, for example, with the inner shaft member 36 extending through a center of the valve prosthesis 10. The outer sheath 126 comprises a cross-sectional size (e.g., diameter) that is less than a cross-sectional size of the valve prosthesis 10 when the valve prosthesis 10 is in a radially-expanded position (e.g., illustrated in FIG. 1). As such, the outer sheath 126, along with the coil 701, can maintain the valve prosthesis 10 in the radially-compressed position during delivery.

Minimally invasive percutaneous interventional procedures, including endovascular procedures, require access to the venous or arterial system. In general, it is desirable to make the smallest incision point with the shortest tissue contact time when entering the body. Small incisions and short tissue contact time generally lead to improved patient outcomes, less complications, and less trauma to the vessels or organs being accessed, as well as less trauma to the skin and tissue through which the access point is created. Access is required for various medical procedures that deliver or implant structural elements (such as heart valves, heart valve repair devices, occluders, grafts, electrical stimulators, leads, etc.) percutaneously. Some procedures employ relatively large devices that require relatively large sheaths to deliver the devices to the intended site within the body. With such procedures, access site trauma can occur, often resulting in vessel damage, excessive bleeding, increased case time, increased risk of infection, and increased hospitalization time. To reduce access trauma, physicians try to use the smallest devices possible and place the smallest sheath size. This can be problematic, however, if during the procedure the physician discovers a larger device is needed. This leads to a need to upsize the sheath, which is a lengthy procedure and leads to increased risk to the patient. Expandable sheaths can be expanded within the body and thus do not require removal to upsize.

Expandable sheath designs may be regionally or locally expansive to selectively and temporarily expand when the device is passing through a region of the sheath and to retract or recover when the device is not passing or has already passed through the sheath. Embodiments disclosed herein may be employed with an expandable introducer sheath that may solve these and other issues that contribute to vascular trauma. The expandable introducer sheath is described with respect to percutaneous access for transcatheter heart valve repair or replacement, and it should be understood that one or more features of the expandable introducer sheath may be employed alone or in combination for other medical procedures requiring percutaneous access, including but not limited to placement of stents, angioplasty, removal of arterial or venous calcification, and pre-dilatation or post-dilatation.

Various embodiments disclosed herein may include an introducer sheath that has a selectively expandable diameter to allow for the passage of a relatively larger device therethrough and further is configured to return to its original diameter upon passage of the device. The various embodiments may reduce damage to surrounding tissues by reducing contact with those tissues and by eliminating the need to exchange sheaths of different sizes. As a result, these embodiments can reduce procedure time, vascular trauma, bleeding, and the resulting risk of infection and other complications.

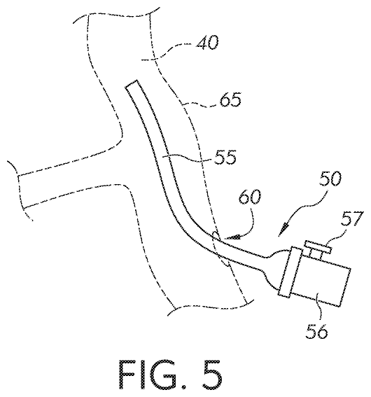

FIG. 5 depicts one embodiment of an introducer sheath 50 positioned through an incision 60 in the skin of a patient and into a vessel 40 (e.g., for example, a femoral vein) surrounded by a vessel wall 65 of a patient. The sheath 50 has a tubular shaft 55 and a proximal hub 56 with a hemostatic seal and a luer lock 57. FIG. 5 shows the sheath 50 positioned in the vessel 40 in its normal, unexpanded state. The shaft 55 can expand as a device passes through and then can retract or recover to its original diameter after the device moves past or is removed from the shaft 55. Thus, the tubular shaft 55 is configured to be expandable and retractable.

In certain embodiments, the expandability of the shaft 55 (and any shaft described according to any embodiment set forth herein) can be achieved via the elasticity of the shaft 55, which can result in the shaft 55 being either expandable or expanding or mechanically expandable or mechanically expanding. For purposes of this application, expandable means that the shaft 55 is configured to expand to a predetermined or nominal diameter. Further, for purposes of this application, mechanically expandable means that the shaft 55 is configured to expand when a positionable medical device is positioned through the shaft 55. That is, the device itself that is being passed through the shaft 55 causes the expansion of the shaft 55. In this way, the expandable and/or mechanically expandable shaft 55 can expand in response to a component or structure being pushed through the shaft 55. Further, in the alternative, while an expandable introducer sheath can be used, in aspects, the transcatheter heart valve prosthesis 10 may be delivered via a non-expandable introducer sheath.

After passage of the device, the shaft 55 is configured to be contractable, retractable, or recoverable to its original, unexpanded state as depicted in FIG. 5. The retractability can be, in certain embodiments, achieved by the elasticity of the shaft 55, which can result in the shaft 55 being either self-retractable or self-retracting, self-recoverable, or self-contractable, or mechanically retractable or mechanically retracting, mechanically recoverable, or mechanically contractable. For purposes of this application, self-retractable means that the shaft 55 is configured to retract to a predetermined or nominal diameter automatically (without any type of actuation, mechanical or otherwise). Further, for purposes of this application, mechanically retractable means that the shaft 55 is configured to retract when a device or component is used to cause the shaft 55 to retract or recover. Alternatively, the retractable characteristics of the shaft 55 can be caused by something other than elasticity.

For purposes of this application, any device that can be positioned through an introducer sheath according to any embodiment disclosed or contemplated herein can be referred to as a positionable medical device or insertable medical device. Such devices include guidewires, dilators, delivery devices (for delivery and/or placement of structural elements such as heart valves, heart valve repair devices, occluders, grafts, electrical stimulators, leads, etc.), guide catheters, guiding sheaths, diagnostic catheters, stent delivery systems, balloon catheters, and other known vascular devices. Other devices can include non-vascular devices such as scopes and other common surgical instruments. Further, the introducer sheath is configured to receive tissues or organs. Thus, as one non-limiting example, the introducer sheath 50 is described as being an expandable introducer sheath 50 for introduction of a delivery assembly 30 including a transcatheter heart valve prosthesis 10.

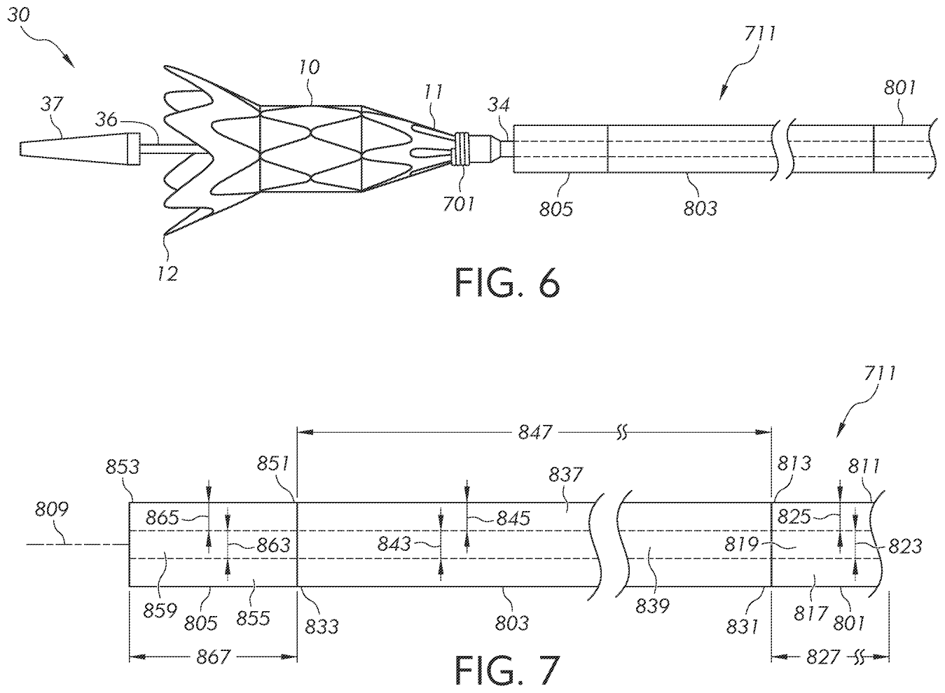

FIG. 6 illustrates a side view of an embodiment of the heart valve prosthesis 10 and a portion of an embodiment of the delivery assembly 30, for example, an inflow capture device 701, the distal tip 37, retractable sheath(s), shaft(s) 34, 36, etc. In aspects, the inflow capture device 701 can comprise a coil that can engage with attachment members 26 (e.g., illustrated in FIG. 1) at the inflow end 11 of the heart valve prosthesis 10, and, thus, support the inflow end 11 in a radially-compressed position. FIG. 6 illustrates the valve prosthesis 10 in the process of deployment, wherein the the outflow end 12 moves from the radially-compressed position to a radially-expanded position. As illustrated in FIG. 6, the inflow capture device 701 is still attached to the inflow end 11, such that the inflow end 11 is in the radially-compressed position. Accordingly, methods of recapturing the heart valve prosthesis 10 can comprise deploying the heart valve prosthesis 10 at the treatment site, wherein the deployment can comprise partial or complete deployment.

In aspects, due to an undesirable position of the valve prosthesis 10 at the treatment site, it may be beneficial to recapture the valve prosthesis 10, for example, by radially-compressing the valve prosthesis 10 from the radially-expanded position to the radially-compressed position. As used herein, the term “recapture” can refer to the process of radially-compressing the valve prosthesis 10 from a partially or completely radially-expanded position to a radially-compressed position, and maintaining the valve prosthesis 10 in the radially-compressed position while repositioning the valve prosthesis 10. As illustrated in FIG. 6, the delivery assembly 30 can comprise a recapture apparatus 711 that is provided to recapture the valve prosthesis 10. In aspects, the recapture apparatus 711 can circumferentially surround portions of the delivery assembly 30, for example, one or more of the inner shaft 36, the holding catheter 34, etc. In this way, while circumferentially surrounding portions of the delivery assembly 30, the recapture apparatus 711 is movable relative to the aforementioned portions of the delivery assembly 30. That is, the recapture apparatus 711 may not be permanently fixed or attached to the delivery assembly 30, such that relative movement between the recapture apparatus 711 and the delivery assembly 30 is provided.

FIG. 7 illustrates a side view of the recapture apparatus 711, wherein portions of the delivery assembly 30 are omitted from view in FIG. 7 so as to more clearly illustrate the recapture apparatus 711. In aspects, the recapture apparatus 711 can comprise a first shaft portion 801, a second shaft portion 803, and a funnel portion 805 positioned end-to-end. For example, the first shaft portion 801 can extend along an axis 809 between a proximal end 811 and a distal end 813. It will be understood that the first shaft portion 801, the second shaft portion 803, and the funnel portion 805 are not limited to extending linearly along the axis 809, and, in operation, one or more of the first shaft portion 801, the second shaft portion 803, and the funnel portion 805 may be bent, curved, or otherwise oriented to extend non-linearly to accommodate for the tortuous anatomy of a patient's vasculature. At least a portion of the first shaft portion 801 can be positioned at an exterior of the patient's vasculature (e.g., outside of the incision 60 as illustrated in FIG. 5). For example, the proximal end 811, and some or all of the length of the first shaft portion 801 from the proximal end 811 toward the distal end 813 can be positioned at the exterior of the patient's vasculature.

The first shaft portion 801 can comprise a first wall 817 surrounding a first chamber 819. In this way, the first shaft portion 801 is substantially hollow, with the first chamber 819 extending along the length of the first shaft portion 801 between the proximal end 811 and the distal end 813. The first chamber 819 can comprise a first chamber diameter 823 that is a distance between an inner surface of the first wall 817 measured in a direction substantially perpendicular to the axis 809. In aspects, the first chamber diameter 823 may be within a range from about 5 millimeters to about 10 millimeters. In aspects, the first chamber diameter 823 may be substantially constant along the length of the first shaft portion 801 between the proximal end 811 and the distal end 813. In this way, portions of the delivery assembly 30 can be received within the first chamber 819. That is, the first shaft portion 801 can receive a first portion of the delivery assembly 30 within the first chamber 819. In aspects, the first wall 817 of the first shaft portion 801 can comprise a first wall thickness 825 that is within a range from about 0.4 millimeters to about 0.8 millimeters. The first wall thickness 825 can be measured in a radial direction between an inner surface of the first wall 817 and an outer surface of the first wall 817. In aspects, the first wall thickness 825 can be substantially constant along the length of the first shaft portion 801 between the proximal end 811 and the distal end 813.

The first shaft portion 801 can comprise several materials, for example, a thermoplastic elastomer such as polyether block amide, a thermoplastic polyurethane elastomer, etc. The first wall 817 can comprise a first durometer value (e.g., shore durometer) that is a measure of the hardness of the first wall 817. In aspects, the first wall 817 can comprise a higher durometer value (e.g., a harder and less flexible material) than the second shaft portion 803 and/or the funnel portion 805. For example, the first durometer of the first wall 817 allows the first shaft portion 801 to be moved in an axial direction along the axis 809 (e.g., toward and away from the incision 60), such that the first shaft portion 801 can apply a pushing force to the second shaft portion 803. In aspects, the first shaft portion 801 can comprise a first length 827 measured between the proximal end 811 and the distal end 813, wherein the first length 827 may be greater than 50 centimeters, or greater than 100 centimeters, or greater than 100 centimeters. In aspects, the first shaft portion 801 can further comprise one or more structures to provide additional reinforcement to enhance the ability to move axially (e.g., pushability) at a minimal dimensional increase. For example, the first shaft portion 801 can be reinforced with one or more of a frame (e.g., a nitinol frame, for example), a braid (e.g., braided sheath, sleeve, etc.), or a coil (e.g., a nitinol coil, for example). In aspects, when the first shaft portion 801 is reinforced with one of these structures, the structure (e.g., frame, braid, coil, etc.) may be embedded within and/or surrounded by the thermoplastic elastomer material described above.

The second shaft portion 803 can be attached to the first shaft portion 801 and, in aspects, can extend along the axis 809 between a proximal end 831 and a distal end 833. The proximal end 831 of the second shaft portion 803 can be attached to the distal end 813 of the first shaft portion 801 such that the first shaft portion 801 and the second shaft portion 803 can be attached and positioned in an end-to-end configuration. In aspects, some, or all, of the second shaft portion 803 can be positioned within the patient's vasculature, for example, with the distal end 833 and some, or all, of the length of the second shaft portion 803 from the distal end 833 toward the proximal end 831 positioned within the patient's vasculature.

The second shaft portion 803 can comprise a second wall 837 surrounding a second chamber 839. In this way, the second shaft portion 803 is substantially hollow, with the second chamber 839 extending along the length of the second shaft portion 803 between the proximal end 831 and the distal end 833. In aspects, the second chamber 839 is contiguous and coaxial with the first chamber 819. The second chamber 839 can comprise a second chamber diameter 843 that is a distance between an inner surface of the second wall 837 measured in a direction substantially perpendicular to the axis 809. In aspects, the second chamber diameter 843 may be within a range from about 5 millimeters to about 10 millimeters. In aspects, the second chamber diameter 843 may be substantially constant along the length of the second shaft portion 803 between the proximal end 831 and the distal end 833. In this way, portions of the delivery assembly 30 can be received within the second chamber 839. That is, the second shaft portion 803 can receive a second portion of the delivery assembly 30 within the second chamber 839. In aspects, the second wall 837 of the second shaft portion 803 can comprise a second wall thickness 845 that is within a range from about 0.4 millimeters to about 0.8 millimeters. The second wall thickness 845 can be measured in a radial direction between an inner surface of the second wall 837 and an outer surface of the second wall 837. In aspects, the second wall thickness 845 can be substantially constant along the length of the second shaft portion 803 between the proximal end 831 and the distal end 833.

The second shaft portion 803 can comprise several materials, for example, one or more of a thermoplastic elastomer material or a thermoplastic polyurethane elastomer. In aspects, the second shaft portion 803 can be reinforced with a nitinol frame or coil while still being capable of a relatively tight bend radius. The second shaft portion 803 can comprise a mid-durometer polymer material. The second wall 837 can comprise a second durometer value (e.g., shore durometer) that is a measure of the hardness of the second wall 837. In aspects, the second durometer value of the second shaft portion 803 is less than the first durometer value of the first shaft portion 801, such that the second wall 837 comprises a softer and more clastic or flexible material than the first wall 817. In this way, the second durometer value of the second shaft portion 803 may be chosen to allow for the second shaft portion 803 to bend and flex while moving through tortuous sections of the patient's vasculature. The second durometer value of the second shaft portion 803 can also allow for the second shaft portion 803 to maintain sufficient axial strength and rigidity to allow for the first shaft portion 801 to apply an axial pushing force upon the second shaft portion 803, thus allowing for the second shaft portion 803 to move axially through the patient's vasculature. In aspects, the second shaft portion 803 can comprise a second length 847 measured between the proximal end 831 and the distal end 833, wherein the second length 847 may be greater than 50 centimeters, or greater than 100 centimeters, or greater than 100 centimeters. In aspects, the second shaft portion 803 can be reinforced with a structure, such that the second shaft portion 803 may, for example, be reinforced with one or more of a frame or a coil comprising nitinol. Reinforcing the second shaft portion 803 (e.g., with the nitinol frame or coil, etc.) can further accommodate for the relatively tight bends within a patient's anatomy. In aspects, when the second shaft portion 803 is reinforced with the nitinol frame or coil, the nitinol frame or coil may be embedded within and/or surrounded by the mid-durometer polymer material described above.

The funnel portion 805 can be attached to the second shaft portion 803 such that the second shaft portion 803 is positioned axially between the first shaft portion 801 and the funnel portion 805. The first shaft portion 801, the second shaft portion 803, and the funnel portion 805 can be attached to one another in several ways, for example, with adhesives or the like. The funnel portion 805 can be attached to the second shaft portion 803 and, in aspects, can extend along the axis 809 between a proximal funnel end 851 and a distal funnel end 853. For example, the proximal funnel end 851 can be attached to the distal end 833 of the second shaft portion 803 such that the funnel portion 805 and the second shaft portion 803 can be attached and positioned in an end-to-end configuration.

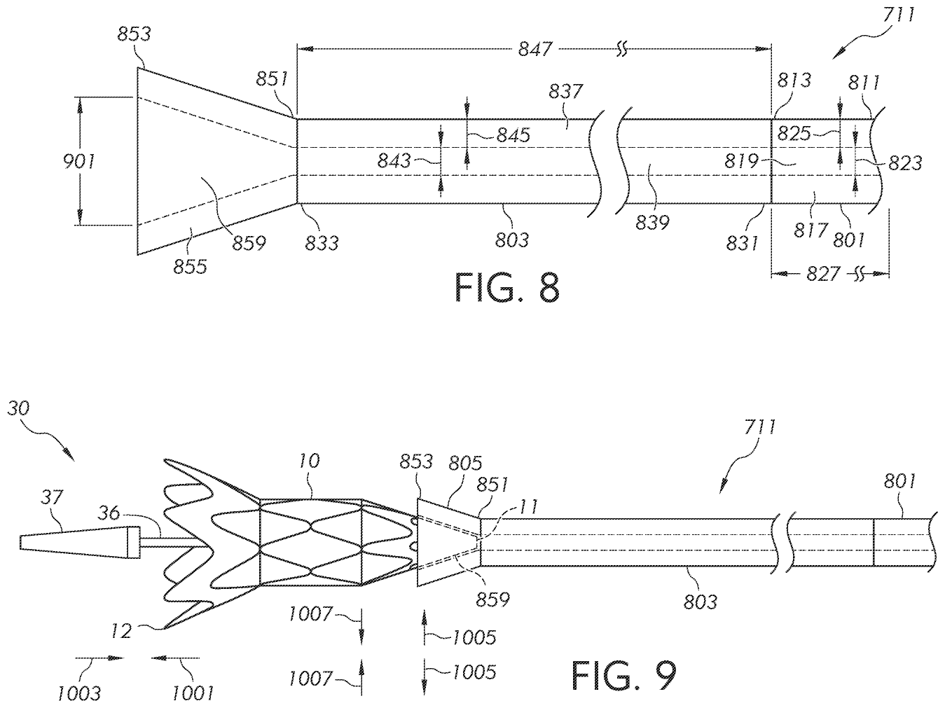

The funnel portion 805 can comprise a funnel wall 855 surrounding a funnel chamber 859. In this way, the funnel portion 805 is substantially hollow, with the funnel chamber 859 extending along the length of the funnel portion 805 between the proximal funnel end 851 and the distal funnel end 853. In aspects, the funnel chamber 859 is contiguous and coaxial with the second chamber 839. For example, the first chamber 819, the second chamber 839, and the funnel chamber 859 can extend coaxially and contiguously through the first shaft portion 801, the second shaft portion 803, and the funnel portion 805. In this way, the first chamber 819, the second chamber 839, and the funnel chamber 859 can receive at least a portion of the delivery assembly 30 (e.g., shafts 34, 36, etc.) through the chambers 819, 839, 859 of the first shaft portion 801, the second shaft portion 803, and the funnel portion 805. The funnel portion 805 can move between a radially-compressed position (e.g., as illustrated in FIG. 7), in which the funnel chamber 859 comprises a first diameter 863 that is less than or equal to the second chamber diameter 843 of the second chamber 839, and a radially-expanded position (e.g., as illustrated in FIG. 8). The funnel portion 805 may be biased into the radially-compressed position and is configured to receive and compress the heart valve prosthesis 10 within the funnel chamber 859. In aspects, the first diameter 863 is the distance between an inner surface of the funnel wall 855 measured in a direction substantially perpendicular to the axis 809. The first diameter 863 (e.g., the cross-sectional size of the funnel chamber 859) may, in aspects, be substantially constant along the length of the funnel portion 805 between the ends 851, 853 when the funnel portion 805 is in the radially-compressed position. In aspects, the first diameter 863 may be within a range from about 5 millimeters to about 10 millimeters. In aspects, the funnel wall 855 can comprise a funnel wall thickness 865 that is within a range from about 0.3 millimeters to about 0.6 millimeters. The funnel wall thickness 865 can be measured in a radial direction between an inner surface of the funnel wall 855 and an outer surface of the funnel wall 855. In aspects, the funnel wall thickness 865 may be substantially constant along the length of the funnel portion 805 between the ends 851, 853.

The funnel portion 805 can comprise, for example, one or more of a polyether-based thermoplastic polyurethane material or a thermoplastic elastomer material. In aspects, the funnel portion 805 can comprise a super-elastic material that can reversibly deform to a high strain in response to a high stress. The funnel wall 855 can comprise a third durometer value (e.g., shore durometer) that is a measure of the hardness of the funnel wall 855. In aspects, the third durometer value of the funnel portion 805 is less than the second durometer value of the second shaft portion 803. As such, the funnel wall 855 comprises a softer and more clastic or flexible material than the first wall 817 and the second wall 837. The funnel portion 805 can therefore be attached to the shaft portions 801, 803, with the funnel portion 805 comprising a different, and softer more flexible material than the shaft portions 801, 803. In this way, the third durometer value of the funnel portion 805 can be chosen to allow for the funnel portion 805 to flex and move between the radially-compressed position and the radially-expanded position. The third durometer value can also allow for the funnel portion 805 to maintain sufficient axial strength and rigidity to allow for the second shaft portion 803 to apply an axial pushing force upon the funnel portion 805, thus allowing for the funnel portion 805 to move axially through the patient's vasculature. In aspects, the funnel portion 805 can comprise a funnel length 867 measured between the proximal funnel end 851 and the distal funnel end 853, wherein the funnel length 867 is within a range from about 5 millimeters to about 10 millimeters.

FIG. 8 illustrates the funnel portion 805 in the radially-expanded position, wherein the funnel chamber 859 comprises a second diameter 901 that is greater than the first diameter 863. In aspects, the second diameter 901 is measured at the distal funnel end 853, which can represent a location of a maximum cross-sectional size (e.g., diameter) of the funnel chamber 859. In aspects, the second diameter 901 may be within a range from about 12 millimeters to about 20 millimeters. When the funnel portion 805 is in the radially-expanded position, the funnel portion 805 may comprise a non-constant and increasing cross-sectional size (e.g., diameter) from the proximal funnel end 851 to the distal funnel end 853. In aspects, the funnel chamber 859 at the proximal funnel end 851 may comprise the first diameter 863 while the funnel chamber 859 at the distal funnel end 853 may comprise the second diameter 901.

As mentioned above, the funnel portion 805 may be biased into the radially-compressed position. As used herein, the term ‘biased’ can refer to the funnel portion 805 reverting back to the radially-compressed position illustrated in FIG. 7 in the absence of other forces acting upon the funnel portion 805, and remaining in the radially-compressed position. However, in response to an outward radial force acting upon the inner surface of the funnel wall 855, the funnel portion 805 can move from the radially-compressed position to the radially-expanded position. In aspects, the outward radial force can occur when the heart valve prosthesis 10 is received within the funnel chamber 859 and contacts the inner surface of the funnel wall 855. In the radially-compressed position, the funnel portion 805 can comprise a minimum cross-sectional size (e.g., diameter) and in the radially-expanded position, the funnel portion 805 can comprise a maximum cross-sectional size. While in the radially-compressed position, the funnel portion 805 can comprise an outer diameter (e.g., a diameter measured at the outer surface of the funnel wall 855) that can substantially match an outer diameter of the second shaft portion 803. In this way, the junction between the second shaft portion 803 and the funnel portion 805 may be relatively smooth to limit the likelihood of negatively affecting the patient's vasculature during movement of the recapture apparatus 711. However, in the radially-expanded position, the outer diameter of the funnel portion 805 may be greater than an outer diameter of the second shaft portion 803, with the outer diameter of the funnel portion 805 increasing from the proximal funnel end 851 to the distal funnel end 853.

Referring to FIGS. 6 and 9, the operation of the recapture apparatus 711 relative to the heart valve prosthesis 10 is illustrated. Initially, as illustrated in FIG. 6, the heart valve prosthesis 10 may be at least partially deployed, for example, with the outflow end 12 in a radially-expanded position and the inflow end 11 in the radially-compressed position (e.g., attached to the inflow capture device 701). However, a physician may determine that the heart valve prosthesis 10 is in an undesirable position relative to the treatment site of the patient. Accordingly, the recapture apparatus 711 can receive and radially compress the heart valve prosthesis 10, allowing for the heart valve prosthesis 10 to be moved and re-deployed. For example, the recapture apparatus 711 may be moved relative to the delivery assembly 30 and the heart valve prosthesis 10 such that the distal funnel end 853 of the funnel portion 805 can be positioned adjacent to the inflow end 11 of the heart valve prosthesis 10 (e.g., as illustrated in FIG. 6). In aspects, movement of the recapture apparatus 711 relative to the delivery assembly 30 can comprise axial movement of the recapture apparatus 711 and/or rotational movement of the recapture apparatus 711. Accordingly, methods can comprise positioning the funnel portion 805 of the heart valve implant recapture apparatus 711 adjacent to the valve end (e.g., inflow end 11) of the heart valve prosthesis 10 with the funnel portion 805 in the radially-compressed position (e.g., illustrated in FIG. 6).

Referring to FIG. 9, the recapture apparatus 711 can be moved from the position illustrated in FIG. 6 in a first movement direction 1001 toward the heart valve prosthesis 10. The recapture apparatus 711 can be moved in the first movement direction 1001, for example, by a physician applying an axial pushing force to the first shaft portion 801, which can likewise cause the second shaft portion 803 and the funnel portion 805 to move axially in the first movement direction 1001. In addition, or in the alternative, the heart valve prosthesis 10 can be moved in a second movement direction 1003 toward the recapture apparatus 711, wherein the second movement direction 1003 is opposite the first movement direction 1001. The recapture apparatus 711 and/or the heart valve prosthesis 10 can be moved relative to one another such that the heart valve prosthesis 10 can be received within the funnel chamber 859.

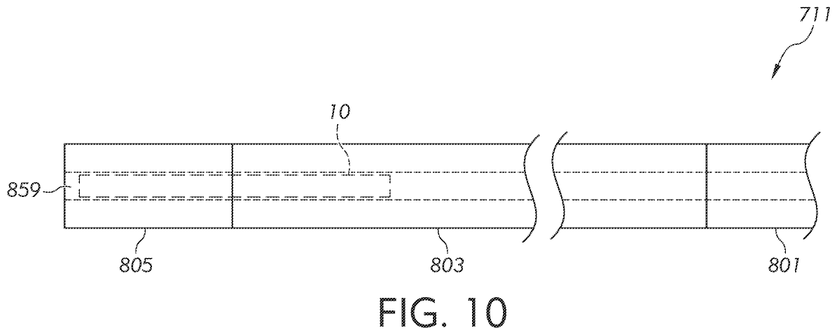

In aspects, the heart valve prosthesis 10 can contact the inner surface of the funnel wall 855 and apply an outward radial force 1005 (e.g., illustrated with a directional arrow) to the funnel wall 855, thus causing the funnel portion 805 to radially expand from the radially-compressed position. Likewise, the funnel wall 855 can apply an inward radial force 1007 (e.g., illustrated with a directional arrow) to the heart valve prosthesis 10, thus causing the heart valve prosthesis 10 to radially contract and move from the radially-expanded position to a radially-compressed position. Methods can therefore comprise moving one or more of the funnel portion 805 (e.g., in the first movement direction 1001) relative to the heart valve prosthesis 10 or the heart valve prosthesis 10 (e.g., in the second movement direction 1003) relative to the funnel portion 805 such that the funnel portion 805 receives the heart valve prosthesis 10 within the funnel chamber 859 and the funnel portion 805 moves from the radially-compressed position to the radially-expanded position. Moving the funnel portion 805 can comprise applying the axial force to the funnel portion 805 by the second shaft portion 803 that is attached to the funnel portion 805, with the funnel portion 805 comprising the durometer value that is less than the durometer value of the second shaft portion 803. Methods can further comprise applying the outward radial force 1005 from the valve prosthesis 10 to the funnel portion 805 to move the funnel portion 805 from the radially-compressed position to the radially-expanded position. The diameter of the funnel chamber 859 can therefore change from the radially-compressed position to the radially-expanded position to accommodate the heart valve prosthesis 10. The recapture apparatus 711 can continue moving in the first movement direction 1001 and/or the heart valve prosthesis 10 can continue moving in the second movement direction 1003 at least until the heart valve prosthesis 10 is received within the funnel chamber 859 (e.g., as illustrated in FIGS. 9-10).

FIG. 10 illustrates the heart valve prosthesis 10 fully received within the funnel chamber 859, such that the length of the heart valve prosthesis 10 is circumferentially surrounded by the funnel wall 855. In this way, the funnel portion 805 can apply the inward radial force 1007 to the heart valve prosthesis 10, thus maintaining the heart valve prosthesis 10 in the radially-compressed position illustrated in FIG. 10. Accordingly, the heart valve prosthesis 10 can be fully recaptured and repositioned, for example, by moving the delivery assembly 30. Methods can therefore comprise applying a radial force (e.g., the inward radial force 1007) from the funnel portion 805 to the heart valve prosthesis 10 to radially compress the heart valve prosthesis 10. The recapture apparatus 711 can provide several benefits. For example, the recapture apparatus 711 can be moved relative to the delivery assembly 30, such that modifications to an existing delivery assembly 30 may be avoided. Further, the recapture apparatus 711 can be used to recapture the heart valve prosthesis 10 and allow for a more desirable positioning of the heart valve prosthesis 10 within the patient's vasculature.

Aspect 1. A transcatheter heart valve delivery assembly is provided for delivering a heart valve prosthesis to a treatment site. The transcatheter heart valve delivery assembly comprises a first shaft portion comprising a first wall surrounding a first chamber and extending along an axis. The first wall comprises a first durometer value. The transcatheter heart valve delivery assembly comprises a second shaft portion attached to a distal end of the first shaft portion and comprising a second wall surrounding a second chamber. The second shaft portion extends along the axis. The second wall comprises a second durometer value that is less than the first durometer value. The transcatheter heart valve delivery assembly comprises a funnel portion attached to a distal end of the second shaft portion and comprising a funnel wall surrounding a funnel chamber. The funnel portion extends along the axis and can move between a radially-compressed position, in which the funnel chamber comprises a first diameter that is less than or equal to a diameter of the second chamber, and a radially-expanded position, in which the funnel chamber comprises a second diameter that is greater than the first diameter. The funnel wall comprises a third durometer value that is less than the second durometer value. The funnel portion is biased into the radially-compressed position and configured to receive and compress the heart valve prosthesis within the funnel chamber.

Aspect 2. The transcatheter heart valve delivery assembly of aspect 1, wherein the funnel portion comprises one or more of a polyether-based thermoplastic polyurethane material or a thermoplastic elastomer material.

Aspect 3. The transcatheter heart valve delivery assembly of any one of aspects 1-2, wherein the funnel wall comprises a funnel wall thickness within a range from about 0.3 millimeters to about 0.6 millimeters.

Aspect 4. The transcatheter heart valve delivery assembly of any one of aspects 1-3, wherein the first diameter is within a range from about 5 millimeters to about 10 millimeters and the second diameter is within a range from about 12 millimeters to about 20 millimeters.

Aspect 5. The transcatheter heart valve delivery assembly of any one of aspects 1-4, wherein the funnel portion comprises a length between a distal funnel end of the funnel portion and a proximal funnel end of the funnel portion that is within a range from about 5 millimeters to about 10 millimeters.

Aspect 6. The transcatheter heart valve delivery assembly of any one of aspects 1-5, wherein the second shaft portion is reinforced with one or more of a frame or a coil.

Aspect 7. The transcatheter heart valve delivery assembly of any one of aspects 1-6, wherein the second wall comprises a second wall thickness within a range from about 0.4 millimeters to about 0.8 millimeters.

Aspect 8. The transcatheter heart valve delivery assembly of any one of aspects 1-7, wherein the second chamber comprises a second chamber diameter within a range from about 5 millimeters to about 10 millimeters.

Aspect 9. The transcatheter heart valve delivery assembly of any one of aspects 1-8, wherein the first shaft portion is reinforced with one or more of a frame, a braid, or a coil.

Aspect 10. The transcatheter heart valve delivery assembly of any one of aspects 1-9, wherein the first chamber, the second chamber, and the funnel chamber extend coaxially and contiguously through the first shaft portion, the second shaft portion, and the funnel portion, the first chamber, the second chamber, and the funnel chamber configured to receive at least a portion of the delivery assembly through the first shaft portion, the second shaft portion, and the funnel portion.

Aspect 11. A transcatheter heart valve delivery assembly is provided for delivering a heart valve prosthesis to a treatment site. The transcatheter heart valve delivery assembly comprises a first shaft portion comprising a first wall surrounding a first chamber. The first wall comprises a first durometer value. The first shaft portion is configured to receive a first portion of the delivery assembly within the first chamber. The transcatheter heart valve delivery assembly comprises a second shaft portion attached to a distal end of the first shaft portion and comprising a second wall surrounding a second chamber. The second wall comprises a second durometer value that is less than the first durometer value. The second shaft portion is configured to receive a second portion of the delivery assembly within the second chamber. The transcatheter heart valve delivery assembly comprises a funnel portion attached to a distal end of the second shaft portion and comprising a funnel wall surrounding a funnel chamber. The funnel portion is configured to move between a radially-compressed position, in which a distal end of the funnel portion comprises a first diameter, and a radially-expanded position, in which the distal end of the funnel portion comprises a second diameter that is greater than the first diameter. The funnel wall comprises a third durometer value that is less than the second durometer value. The funnel portion is biased into the radially-compressed position.

Aspect 12. The transcatheter heart valve delivery assembly of aspect 11, wherein the funnel portion comprises one or more of a polyether-based thermoplastic polyurethane material or a thermoplastic elastomer material.

Aspect 13. The transcatheter heart valve delivery assembly of any one of aspects 11-12, wherein the funnel wall comprises a funnel wall thickness within a range from about 0.3 millimeters to about 0.6 millimeters.

Aspect 14. The transcatheter heart valve delivery assembly of any one of aspects 11-13, wherein the second shaft portion comprises one or more of a thermoplastic elastomer material or a thermoplastic polyurethane elastomer.

Aspect 15. The transcatheter heart valve delivery assembly of any one of aspects 11-14, wherein the second wall comprises a second wall thickness within a range from about 0.4 millimeters to about 0.8 millimeters.

Aspect 16. Methods of recapturing a heart valve prosthesis comprise deploying the heart valve prosthesis at a treatment site. Methods comprise positioning a funnel portion of a heart valve implant recapture apparatus adjacent to a valve end of the heart valve prosthesis with the funnel portion in a radially-compressed position. Methods comprise moving one or more of the funnel portion relative to the heart valve prosthesis or the heart valve prosthesis relative to the funnel portion such that the funnel portion receives the heart valve prosthesis within the funnel chamber and the funnel portion moves from the radially-compressed position to a radially-expanded position. Methods comprise applying a radial force from the funnel portion to the heart valve prosthesis to radially compress the heart valve prosthesis.

Aspect 17. The method of aspect 16, wherein the moving the funnel portion comprises applying an axial force to the funnel portion by a second shaft portion that is attached to the funnel portion, the funnel portion comprising a durometer value that is less than the second shaft portion.

Aspect 18. The method of any one of aspects 16-17, further comprising applying an outward radial force from the heart valve prosthesis to the funnel portion to move the funnel portion from the radially-compressed position to a radially-expanded position.

Aspect 19. The method of any one of aspects 16-18, wherein a diameter of the funnel chamber changes from the radially-compressed position to the radially-expanded position.

Aspect 20. The method of any one of aspects 16-19, wherein the funnel portion is attached to a shaft portion that comprises a different material than the funnel portion.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A transcatheter heart valve delivery assembly for delivering a heart valve prosthesis to a treatment site, the transcatheter heart valve delivery assembly comprising:
a first shaft portion comprising a first wall surrounding a first chamber and extending along an axis, the first wall comprising a first durometer value;
a second shaft portion attached to a distal end of the first shaft portion and comprising a second wall surrounding a second chamber, the second shaft portion extending along the axis, the second wall comprising a second durometer value that is less than the first durometer value; and
a funnel portion attached to a distal end of the second shaft portion and comprising a funnel wall surrounding a funnel chamber, the funnel portion extending along the axis and configured to move between a radially-compressed position, in which the funnel chamber comprises a first diameter that is less than or equal to a diameter of the second chamber, and a radially-expanded position, in which the funnel chamber comprises a second diameter that is greater than the first diameter, the funnel wall comprising a third durometer value that is less than the second durometer value, the funnel portion biased into the radially-compressed position and configured to receive and compress the heart valve prosthesis within the funnel chamber.

2. The transcatheter heart valve delivery assembly of claim 1, wherein the funnel portion comprises one or more of a polyether-based thermoplastic polyurethane material or a thermoplastic elastomer material.

3. The transcatheter heart valve delivery assembly of claim 1, wherein the funnel wall comprises a funnel wall thickness within a range from about 0.3 millimeters to about 0.6 millimeters.

4. The transcatheter heart valve delivery assembly of claim 1, wherein the first diameter is within a range from about 5 millimeters to about 10 millimeters and the second diameter is within a range from about 12 millimeters to about 20 millimeters.

5. The transcatheter heart valve delivery assembly of claim 1, wherein the funnel portion comprises a length between a distal funnel end of the funnel portion and a proximal funnel end of the funnel portion that is within a range from about 5 millimeters to about 10 millimeters.

6. The transcatheter heart valve delivery assembly of claim 1, wherein the second shaft portion is reinforced with one or more of a frame or a coil.

7. The transcatheter heart valve delivery assembly of claim 1, wherein the second wall comprises a second wall thickness within a range from about 0.4 millimeters to about 0.8 millimeters.

8. The transcatheter heart valve delivery assembly of claim 1, wherein the second chamber comprises a second chamber diameter within a range from about 5 millimeters to about 10 millimeters.

9. The transcatheter heart valve delivery assembly of claim 1, wherein the first shaft portion is reinforced with one or more of a frame, a braid, or a coil.

10. The transcatheter heart valve delivery assembly of claim 1, wherein the first chamber, the second chamber, and the funnel chamber extend coaxially and contiguously through the first shaft portion, the second shaft portion, and the funnel portion, such that the first chamber, the second chamber, and the funnel chamber are configured to receive at least a portion of the delivery assembly through the first shaft portion, the second shaft portion, and the funnel portion.

11. A transcatheter heart valve delivery assembly for delivering a heart valve prosthesis to a treatment site, the transcatheter heart valve delivery assembly comprising:
a first shaft portion comprising a first wall surrounding a first chamber, the first wall comprising a first durometer value, the first shaft portion configured to receive a first portion of the delivery assembly within the first chamber;
a second shaft portion attached to a distal end of the first shaft portion and comprising a second wall surrounding a second chamber, the second wall comprising a second durometer value that is less than the first durometer value, the second shaft portion configured to receive a second portion of the delivery assembly within the second chamber; and
a funnel portion attached to a distal end of the second shaft portion and comprising a funnel wall surrounding a funnel chamber, the funnel portion configured to move between a radially-compressed position, in which a distal end of the funnel portion comprises a first diameter, and a radially-expanded position, in which the distal end of the funnel portion comprises a second diameter that is greater than the first diameter, the funnel wall comprising a third durometer value that is less than the second durometer value, the funnel portion biased into the radially-compressed position.

12. The transcatheter heart valve delivery assembly of claim 11, wherein the funnel portion comprises one or more of a polyether-based thermoplastic polyurethane material or a thermoplastic elastomer material.

13. The transcatheter heart valve delivery assembly of claim 11, wherein the funnel wall comprises a funnel wall thickness within a range from about 0.3 millimeters to about 0.6 millimeters.

14. The transcatheter heart valve delivery assembly of claim 11, wherein the second shaft portion comprises one or more of a thermoplastic elastomer material or a thermoplastic polyurethane elastomer.

15. The transcatheter heart valve delivery assembly of claim 11, wherein the second wall comprises a second wall thickness within a range from about 0.4 millimeters to about 0.8 millimeters.

16. A method of recapturing a heart valve prosthesis comprising:
deploying the heart valve prosthesis at a treatment site;
positioning a funnel portion of a heart valve implant recapture apparatus adjacent to a valve end of the heart valve prosthesis with the funnel portion in a radially-compressed position;
moving one or more of the funnel portion relative to the heart valve prosthesis or the heart valve prosthesis relative to the funnel portion such that the funnel portion receives the heart valve prosthesis within a funnel chamber of the funnel and the funnel portion moves from the radially-compressed position to a radially-expanded position; and
applying a radial force from the funnel portion to the heart valve prosthesis to radially compress the heart valve prosthesis.

17. The method of claim 16, wherein the moving the funnel portion comprises applying an axial force to the funnel portion by a second shaft portion that is attached to the funnel portion, the funnel portion comprising a durometer value that is less than the second shaft portion.

18. The method of claim 16, further comprising applying an outward radial force from the heart valve prosthesis to the funnel portion to move the funnel portion from the radially-compressed position to a radially-expanded position.

19. The method of claim 16, wherein a diameter of the funnel chamber changes from the radially-compressed position to the radially-expanded position.

20. The method of claim 16, wherein the funnel portion is attached to a shaft portion that comprises a different material than the funnel portion.
